(19) United States
(12) Patent Application Publication
(10) Pub. No.: US 2006/0264716 A1
(43) Pub. Date: Nov. 23, 2006

(54) MICRONEEDLE WITH GLUCOSE SENSOR AND METHODS THEREOF

(76) Inventor: Dennis Zander, Penfield, NY (US)

Correspondence Address: JAECKLE FLEISCHMANN & MUGEL, LLP, 190 Linden Oaks, ROCHESTER, NY, 14625-2812, US

(21) Appl. No.: 11/380,063

(22) Filed: Apr. 25, 2006

Related U.S. Application Data
(60) Provisional application No. 60/674,465, filed on Apr. 25, 2005.

(51) Int. Cl. A61B 5/00

(52) U.S. Cl. 600/309; 29/25.41; 29/854

(57) ABSTRACT

A method for making a needle for monitoring blood, a blood monitoring system and a needle array system for monitoring blood are described. Sensors are associated with each microneedle so that each microneedle can sample blood chemistry without extraction. The sensing process is achieved while the needle is inside the patient, minimizing invasiveness and contamination.

### CROSS REFERENCE TO RELATED APPLICATIONS

This application claims the benefit of U.S. Provisional Application No. 60/674,465 filed Apr. 25, 2005, the entire disclosure of which is incorporated herein by reference.

### GOVERNMENT SPONSORSHIP

This invention was made with government support under Grant NAG3-2744 awarded by NASA. The Government has certain rights in the invention.

### BACKGROUND

Microneedle technology provides a useful minimally-invasive method to sample blood. Due to their small size, microneedles can pierce skin and take minute quantities of blood with minimal impact and or pain to the subject. In spite of their advantages, prior art microneedle systems are still somewhat invasive since they involve the extraction of blood from the patient.

Implanted in vivo sensors provide another means to sample blood chemistry that does not require blood extraction. Unfortunately, in vivo sensors interact with the physiology and are susceptible to degradation during use. It would be desirable to achieve a less invasive approach that would not extract blood from the patient and provide longer measurement times than in prior art in vivo devices.

### FIELD OF THE INVENTION

This invention relates to a microneedle with a glucose sensor.

### BRIEF DESCRIPTION OF THE DRAWINGS

FIG. 1 is a perspective view of a microneedle with a glucose sensor in accordance with embodiments of the present invention;

FIG. 2 is a side, cross-sectional view of the microneedle with the glucose sensor shown in FIG. 1;

FIG. 3A is a perspective of an array of microneedles with glucose sensors in accordance with embodiments of the present invention;

FIG. 3B is a top view of the array of microneedles with glucose sensors shown in FIG. 3A;

FIG. 4A is a side, cross-sectional view of a reference microneedle;

FIG. 4B is a side, cross-sectional view of a working microneedle; and

FIG. 4C is a side view of another array of microneedles in accordance with embodiments of the present invention.

### SUMMARY

A method for making a needle for monitoring blood, a blood monitoring system and a needle array system for monitoring blood are described. A method for making a needle for monitoring. blood, the method comprises: fabricating one or more needles in a conductive substrate; placing at least one insulating layer on an outer surface of the needles, wherein a tip region of at least one of the needles remains exposed to the conductive substrate; placing at least one conductive layer on the outer surface of the needles, wherein at least a portion of the tip region of at least one of the needles remains exposed to the conductive substrate; and placing at least one sensing layer on the at least a portion of the tip region of at least one of the needles which remains exposed to the conductive substrate and on at least a portion of the conductive layer.

Another embodiment comprises: fabricating one or more needles in a substrate; placing at least one insulating layer on an outer surface of the needles; placing at least one first conductive layer on the outer surface of the needles; placing at least one second conductive layer on a portion of needles; coating the needles with an exposed first conductive layer with at least one catalyst; placing at least one sensing layer on the needles with the catalyst; and placing at least one protective layer over the second conductive layer and the sensing layer.

A needle array system for monitoring blood, the system comprises: one or more conductive needles formed in a conductive substrate; at least one insulating layer on an outer surface of the needles, wherein a tip region of at least one of the needles remains exposed to the conductive substrate; at least one conductive layer on the outer surface of the needles, wherein at least a portion of the tip region of at least one of the needles remains exposed to the conductive substrate; and at least one sensing layer on the at least a portion of the tip 10 region of at least one of the needles which remains exposed to the conductive substrate and on at least a portion of the conductive layer.

A blood monitoring system, the system comprises: one or more needles formed from a substrate; at least one insulating layer on an outer surface of the needles; at least one first conductive layer on the outer surface of the needles; at least one second conductive layer on a portion of needles; at least one catalyst on the needles with an exposed first conductive layer; at least one sensing layer on the needles with the catalyst; and at least one protective layer over the second conductive layer and the sensing layer.

Unlike the prior art, the invention describes sensors associated with each microneedle that can sample blood chemistry without extraction. The sensing process is achieved while the needle is inside the patient, minimizing invasiveness and contamination.

### DETAILED DESCRIPTION

The microneedles described here are fabricated using typical MEMS methodologies. The needles are typically 200 uM tall, 40 uM in diameter at the tip, and much wider at the base. In some embodiments, the needs are no wider at the base than the tip or they are only somewhat wider. Microneedles of this size have been shown to provide access to interstitial body fluid without reaching the capillaries or nerves, so there is no discomfort. There are two types of integrated sensors described. The first has the sensor materials applied to the tip of a single microneedle; however, this could just as easily apply to multiple microneedles.

First Type of Sensor for Microneedle

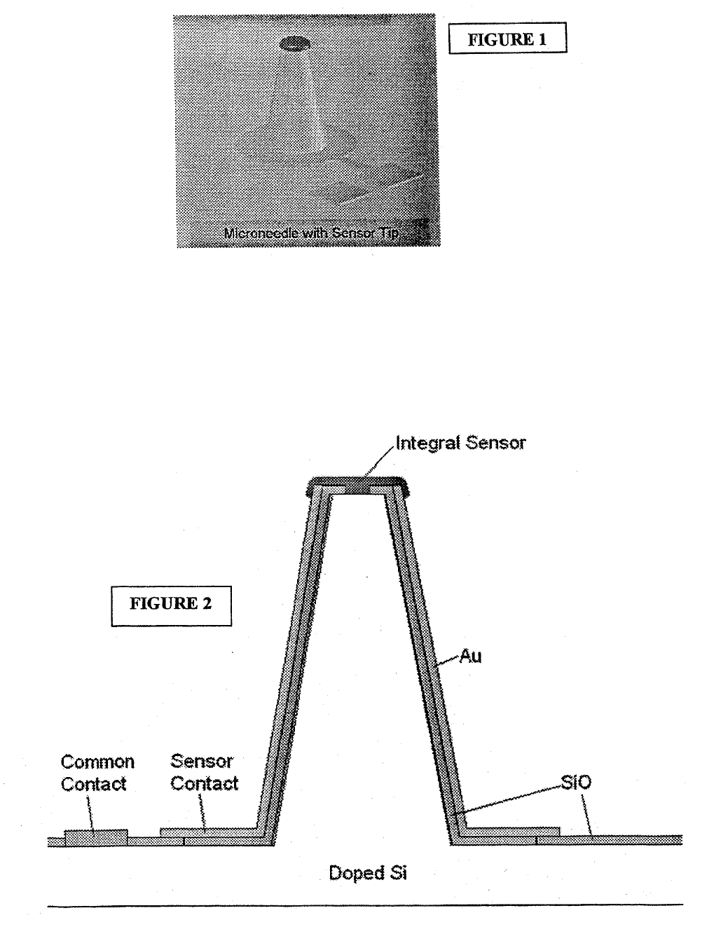

The silicon substrate has one or more microneedles fabricated on its surface. The silicon is doped to make it conductive. Next, a silicon oxide layer is laid down to insulated the surface and the sides of the microneedle, but a small hole is left in the tip of the needle to provide an electrical connection to the doped silicon. Next, a layer of platinum-iridium or gold is put down on each microneedle. This conductive layer is to cover the needle and its immediate base, but not the tip or the surrounding area, so that each needle has an independent electrical connection. Finally, one or more sensing layers are deposited on the tip of the needle. These could be materials like glucose oxidase for the detection of glucose level in interstitial fluids followed by a protective layer to block out common interferences. FIG. 1 shows an isometric view and FIG. 2 shows a section view to show the physical construction and layers.

It is understood that the individual device shown could be replicated on the silicon surface very easily and this could provide redundancy of measurement. Furthermore, the microneedles and sensors could be individual actuated so that as a sensor loses accuracy a new one could be inserted, calibrated to the old one and the then the old one removed.

Second Type of Sensor for Microneedle

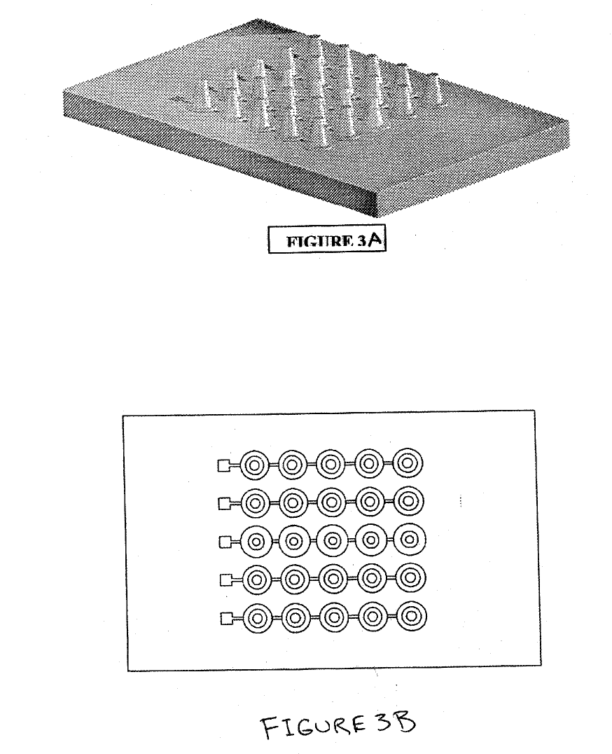

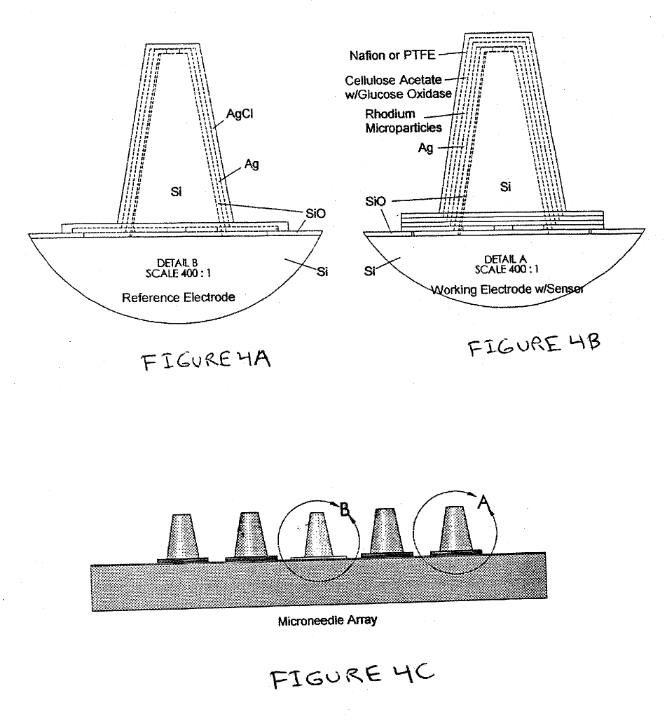

As in the first type, there are multiple needles fabricated from and on a silicon wafer. There is no doping of the silicon required. In this case, the entire surface of the needles and the surface between needles are insulated by a coating of SiO. Then, the individual needles have silver deposited on them. Rows of needles are shown connected together in FIGS. 3A-3B and FIGS. 4A-4C, but they could also be individually sensed and inserted. Then, the reference needles have silver chloride deposited on them. Next, the working needles are coated with rhodium particles for a catalyst. Then, a layer of cellulose acetate and glucose oxidase is put on the working needles. Finally, a layer of Nafion or PTFE is applied to block common interferences. This results in the array of needles as shown in FIGS. 3A-3B. The section view of the structure and coatings is shown in FIGS. 4A-4C.

The reference needles establish a value for an inserted needle which is not sensitive to the analyte of interest. This background value can be used a baseline from which to measure the analyte of interest using the working needles. Having a reference value and working values, permits a more sensitive measurement, since the reference value can be subtracted from or divided into the working value to effectively eliminate body fluid and environmental variations.

Although the present invention has been described in considerable detail with reference to certain preferred versions thereof, other versions are possible. For example, the microneedles can be comprised of glass. Therefore, the spirit and scope of the appended claims should not be limited to the description of the preferred versions contained herein.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features. Additionally, the recited order of processing elements or sequences, or the use of numbers, letters, or other designations therefore, is not intended to limit the claimed processes to any order except as may be specified in the claims.

Any element in a claim that does not explicitly state “means” for performing a specified function or “step” for performing a specified function should not be interpreted as a “means” or “step” clause as specified in 35 U.S.C. §112. Accordingly, the invention is limited only by the following claims and equivalents thereto.

What is claimed is:

1. A method for making a needle for monitoring. blood, the method comprising:
fabricating one or more needles in a conductive substrate;
placing at least one insulating layer on an outer surface of the needles, wherein a tip region of at least one of the needles remains exposed to the conductive substrate;
placing at least one conductive layer on the outer surface of the needles, wherein at least a portion of the tip region of at least one of the needles remains exposed to the conductive substrate; and
placing at least one sensing layer on the at least a portion of the tip region of at least one of the needles which remains exposed to the conductive substrate and on at least a portion of the conductive layer.

2. The method as set forth in claim 1 further comprising doping a substrate to form the conductive substrate.

3. The method as set forth in claim 1 wherein the insulating layer comprises silicon oxide.

4. The method as set forth in claim 1 wherein the conductive layer comprises one of platinum-iridium and gold.

5. The method as set forth in claim 1 wherein the sensing layer comprises:
a layer of glucose oxidase for the detection of glucose level in interstitial fluids; and
a protective layer on the layer of glucose oxidase to block out common interferences.

6. A needle array system for monitoring blood, the system comprising:
one or more conductive needles formed in a conductive substrate;
at least one insulating layer on an outer surface of the needles, wherein a tip region of at least one of the needles remains exposed to the conductive substrate;
at least one conductive layer on the outer surface of the needles, wherein at least a portion of the tip region of at least one of the needles remains exposed to the conductive substrate; and
at least one sensing layer on the at least a portion of the tip region of at least one of the needles which remains exposed to the conductive substrate and on at least a portion of the conductive layer.

7. The system as set forth in claim 6 wherein the conductive substrate is a doped substrate.

8. The system as set forth in claim 6 wherein the insulating layer comprises silicon oxide.

9. The system as set forth in claim 6 wherein the conductive layer comprises one of platinum-iridium and gold.

10. The system asset forth in claim 6 wherein the sensing layer comprises:
a layer of glucose oxidase for the detection of glucose level in interstitial fluids; and;
a protective layer on the layer of glucose oxidase to block out common interferences.

11. A method for making a needle for monitoring blood, the method comprising:
fabricating one or more needles in a substrate;
placing at least one insulating layer on an outer surface of the needles;
placing at least one first conductive layer on the outer surface of the needles;
placing at least one second conductive layer on a portion of needles;
coating the needles with an exposed first conductive layer with at least one catalyst;
placing at least one sensing layer on the needles with the catalyst; and
placing at least one protective layer over the second conductive layer and the sensing layer.

12. The method as set forth in claim 11 wherein the insulating layer comprises silicon oxide.

13. The method as set forth in claim 11 wherein the first conductive layer comprises silver.

14. The method as set forth in claim 11 wherein the second conductive layer comprises silver chloride.

15. The method as set forth in claim 11 wherein the catalyst comprises rhodium particles.

16. The method as set forth in claim 11 wherein the sensing layer of cellulose acetate and glucose oxidase.

17. The method as set forth in claim 11 wherein the protective layer comprises a layer of one of Nafion or PTFE to block common interferences.

18. A blood monitoring system, the system comprising:
one or more needles formed from a substrate;
at least one insulating layer on an outer surface of the needles;
at least one first conductive layer on the outer surface of the needles;
at least one second conductive layer on a portion of needles; at least one catalyst on the needles with an exposed first conductive layer;
at least one sensing layer on the needles with the catalyst; and
at least one protective layer over the second conductive layer and the sensing layer.

19. The system as set forth in claim 18 wherein the insulating layer comprises silicon oxide.

20. The system as set forth in claim 18 wherein the first conductive layer comprises silver.

21. The system as set forth in claim 18 wherein the second conductive layer comprises silver chloride.

22. The system as set forth in claim 18 wherein the catalyst comprises rhodium particles.

23. The system as set forth in claim 18 wherein the sensing layer of cellulose acetate and glucose oxidase.

24. The system as set forth in claim 18 wherein the protective layer comprises a layer of one of Nafion or PTFE to block common interferences.
